(19) United States
(12) Patent Application Publication
(10) Pub. No.: US 2019/0008850 A1
(43) Pub. Date: Jan. 10, 2019

(54) METHOD OF TREATING SCHIZOPHRENIA

(71) Applicant: Paul J. MARKOVITZ, Santa Barbara, CA (US)

(72) Inventor: Paul J. MARKOVITZ, Santa Barbara, CA (US)

(21) Appl. No.: 16/067,046

(22) PCT Filed: Dec. 29, 2016
(86) PCT No.: PCT/US16/69110

§ 371 Date: Jun. 28, 2018

Related U.S. Application Data
(60) Provisional application No. 62/273,247, filed on Dec. 30, 2015.

(51) Int. Cl. A61K 31/485; A61K 31/4515; A61K 31/5415; A61K 31/496; A61K 45/06; A61K 31/4709; A61P 25/18

(52) U.S. Cl. CPC A61K 31/485; A61K 31/4515; A61K 31/5415; A61P 25/18; A61K 45/06; A61K 31/4709; A61K 31/496

(57) ABSTRACT

A method of treating schizophrenia includes parenterally administering to a subject a pharmacological agent that inhibits presynaptic glutamate release, modulates postsynaptic glutamate response, or both.

### RELATED PATENT APPLICATION

This application is the U.S. National Phase of International Application No. PCT/US2016/069110, filed Dec. 29, 2016, which claims the benefit of U.S. Provisional Patent Application No. 62/273,247 filed on Dec. 30, 2015, entitled METHOD OF TREATING SCHIZOPHRENIA, naming Paul J. Markovitz as an inventor. The entire content of the foregoing patent applications are incorporated herein by reference.

### BACKGROUND

The present disclosure generally relates to the field of psychiatric treatment protocols. More specifically, embodiments pertain to methods for treating symptoms of schizophrenia.

Schizophrenia is a debilitating illness with poor longitudinal outcome. Schizophrenia can be described in terms of positive and negative symptoms. Positive symptoms are generally absent in people without schizophrenia and may also be referred to as manifestations of psychosis. Examples of positive symptoms include, without limitation, delusions, disordered thoughts, and hallucinations. Negative symptoms, on the other hand, are those which are more socially based behaviors. Lack of social appropriateness and awareness are present in people with schizophrenia, but not generally present in people without schizophrenia. Examples of negative symptoms include, without limitation, lack of emotion, pleasure, motivation, and desire to form relationships. These symptoms represent social interactions/appropriateness, mood, and temperament and have the largest impact on short and long-term outcome of schizophrenic subjects.

Through conventional treatment protocols, it is possible to reduce positive symptoms of schizophrenia. Although the negative symptoms present in a number of psychiatric disorders may be reversed, there are no known protocols for treating negative symptoms of schizophrenia. Thus, conventional protocols are unable to improve the ability of afflicted individuals to interact socially, work, or develop meaningful relationships. Negative symptoms are the ones that make social interactions, ability to read social cues, display of affection, and ultimately ability to exist in society problematic for individuals with schizophrenia.

Glutamate abnormalities in the brain are believed to be part, even a substantial cause, of schizophrenic symptoms. The current theory behind the cause of negative symptoms in schizophrenia is that there is too little glutamate in certain regions of the brain causing and/or contributing to the negative symptoms. Various medications currently undergoing clinical research trials are aimed at increasing the amount of glutamate in the brains of individuals with schizophrenia. However, these clinical trials have failed to show statistically meaningful improvement. Negative symptoms can also be partially addressed through various forms of cognitive-behavioral therapy, but most individuals have an incomplete response, and very few are able to engage in therapy to garner any benefit at all.

Pseudobulbar affect (“PBA”) is a neurological disorder that can be characterized by involuntary or uncontrollable episodes of laughter or crying, which results from a pathologically lowered threshold for exhibiting these responses. PBA may be present in subjects with multiple neurological diseases causing damage to the central nervous system. Affected individuals may exhibit laughter and/or crying without typical motivating stimuli or in response to stimuli which, for the individual, would not have elicited the response prior to the onset of the neurologic disorder.

The current theory behind PBA is that pathways between the cortical and subcortical structures in the limbic system are damaged by glutamatergic excitotoxicity. If increased glutamate levels are in fact the cause of PBA, then theoretically, treatment protocols which are designed to reduce glutamate would prove beneficial.

In 2010, the Federal Food and Drug Administration (“FDA”) approved the use of an oral drug combination comprising dextromethorphan and quinidine (collectively, “DM/Q”) for the treatment of PBA. It has been indicated that dextromethorphan (“DM”) can influence glutamate signaling through presynaptic inhibition of glutamate release and postsynaptic glutamate response modulation. Quinidine (“Q”) is believed to inhibit liver enzymes that metabolize dextromethorphan allowing therapeutic concentrations of dextromethorphan to cross the blood-brain barrier and interact at brain receptors. Orally administered DM/Q has been shown to be safe and effective in PBA treatment, with minimal side effects, and clinically has been shown to reduce the rate of PBA episodes in subjects suffering with amyotrophic lateral sclerosis and multiple sclerosis. A formulation of DM/Q is commercially available under the trademark NUEDEXTA™, owned by Avanir Pharmaceuticals, Inc.

### SUMMARY

In some aspects, embodiments herein relate to methods of treating schizophrenia includes parenterally administering to a subject a pharmacological agent that inhibits presynaptic glutamate release, modulates postsynaptic glutamate response, or both.

### DETAILED DESCRIPTION

Embodiments herein relate to the use of dextromethorphan, and in particular its use in reducing symptoms of schizophrenia. In connection with the present embodiments, it has been shown that conventional thinking that the cause of negative symptoms in schizophrenia is due to too little glutamate in certain regions of the brain may be wrong. This may explain why medications tested to date that increase glutamate levels do not work to treat schizophrenia in general, and negative symptoms in particular. In contrast, in accordance with the present embodiments, it is more likely that too much glutamate is actually causing negative symptoms of schizophrenia. Accordingly, some embodiments that are provided are treatment protocols which reduce glutamate levels, including but not limited to, by administration of dextromethorphan and dextromethorphan combined with quinidine.

Therapeutic administration of dextromethorphan substantially reduces negative symptoms of schizophrenia. When administered at first break of schizophrenia, it mitigated the progression of schizophrenia, and in some cases, reversed the disorder. Administration to individuals with established schizophrenia diagnoses significantly reduced the frequency and intensity of negative symptoms. Moreover, administration of dextromethorphan appears to also reduce some positive symptoms of schizophrenia, and in particular, expressions of hostility.

Embodiments herein are explained in greater detail below. While the various embodiments are described in conjunction with several examples, these exemplary embodiments themselves are not limiting in scope. Thus, the claims may cover alternatives, modifications, and/or equivalents of the exemplary embodiments.

Although conventional theory is that decreased glutamate levels in the central nervous system contribute to negative symptoms of schizophrenia, the foundational theory of embodiments herein is that increased glutamate levels actually contribute to negative symptoms of schizophrenia. Consequently, the present embodiments challenge conventional thinking in several regards.

There is a continuing need for an accurate animal model for studying schizophrenia. Schizophrenia is believed to be a distinctly human disease. The model used in animals and humans to represent schizophrenia is administration of ketamine, which can result in schizophrenia-like symptoms. Although administration of ketamine can induce psychosis, such psychosis arises from different biological pathways than psychosis arising from schizophrenia. If enough cells are lost in certain areas of the central nervous system, glutamate levels could appear low simply because the cells producing glutamate have died. High levels of glutamate could be neurotoxic, resulting in death of cells and what was too high a level of glutamate is now being read as too low a level due to cell death. Since DM/Q unequivocally decreases glutamate levels, conventional theory about treating negative symptoms of schizophrenia would expect to see a worsening of negative symptoms of schizophrenia with time, however, just the opposite is observed.

Medications tested to date which increase glutamate levels are not effective in treating schizophrenia, in general, and negative symptoms of the disease, in particular. If the pharmacological interventions according to conventional theory being investigated through current Food and Drug Administration approved trials (i.e., increasing glutamate levels in individuals suffering from schizophrenia) are working in an opposite manner of what is needed in the brain of schizophrenic subjects (i.e., increasing glutamate when too much is already present in the brain) and an increased glutamate level is one of the causes of negative symptoms in schizophrenia, these drugs will not work. Moreover, it is possible many individuals could even become worse as glutamate levels increase, because high glutamate levels can be neurotoxic.

Without being bound by theory it is postulated that increased glutamate levels contribute to negative symptoms of schizophrenia such that modulation of glutamate would be an effective treatment protocol. Thus, in accordance with embodiments disclosed herein, a method for treating symptoms of schizophrenia can comprise administering an effective amount of a pharmacological agent that inhibits presynaptic glutamate release and/or modulates postsynaptic glutamate response. In some embodiments, the method can additionally comprise administering a neuroleptic.

In some embodiments, there are provided methods of treating schizophrenia comprising parenterally administering to a subject a pharmacological agent that inhibits presynaptic glutamate release, modulates postsynaptic glutamate response, or both. In some embodiments, the pharmacological agent comprises dextromethorphan. Dextromethorphan (DM) can be provided as any pharmacologically acceptable salt form. In accordance with embodiments herein, DM is substantially the only active ingredient administered to a subject having schizophrenia. By way of example, embodiments herein encompass the therapeutic use of DM to treat symptoms of schizophrenia in the absence of quinidine, which is used when DM is administered orally, as is the case with the product NUDEXTA® (Avanir Pharmaceuticals, Aliso Viejo, Calif.). Without being bound by theory, it is postulated that any pharmacological agent that inhibits presynaptic glutamate release or modulates postsynaptic glutamate response can operate in a manner analogous to parenterally administered DM. For example, other pharmacological agents can include, without limitation, riluzole and lamotrigine and its known derivatives. See Obrenovitch et al., Amino Acids 14:143-150 (1998), which is incorporated herein by reference in its entirety. Although embodiments herein focus primarily on DM as the main active ingredient, other embodiments may provide combinations of DM with riluzole and/or lamotrigine and/or its derivatives.

As used herein “parenterally administering” refers to the use of any non-enteral mode of administration. In some embodiments, parenteral administration may include intravenous, subcutaneous, intramuscular modes and the like. Although these parenteral administration modes are exemplary of non-enteric delivery, those skilled in the art will appreciate further non-enteric modes which may be effective in the methods disclosed herein including, without limitation, mucosal, otic, intrathecal, intracerebral, topical, intraocular, transdermal, and other delivery modes. In some embodiments, there can be mixed modes of administration, such as a combination of oral (or other enteric mode) administration with parenteral modes. Modes of administration can be carried out in conjunction with various devices as understood by those skilled in the art including, without limitation, pumps, patches, and the like.

As used herein “inhibiting presynaptic glutamate release” refers to decreasing the total amount of glutamate released initially by central nervous system cells which then stimulates reactions in other central nervous system cells that are downstream from where the glutamate is initially released. Too much glutamate leads to neurotoxicity, but some release is necessary for normal cell functioning. Lowering the “too high” amount of glutamate released by cells in the brain that start the cascade of events leading to neurotoxicity to a more normal or less pathological level may abate schizophrenic symptoms and inhibit the progression of the illness. Glutamate levels can be measured through magnetic resonance spectroscopy, and as has been demonstrated in a number of psychiatric illnesses.

As used herein “modulating postsynaptic glutamate response” refers to the measurement of reduction in pathological behaviors on the Positive, Negative, and General Psychopathology Symptoms of Schizophrenia Scale. Lowering of negative symptoms is a behavioral measure indicating a modulation of glutamate release to a more normal level. The modulation will also likely include reduction in positive symptoms as well as the general psychopathology score portion of the PANSS, too.

In some embodiments, treating schizophrenia comprises modulating positive symptoms. Positive symptoms include, without limitation, delusions, disordered thoughts, and hallucinations. In some embodiments, treating of schizophrenia comprises modulating negative symptoms. Negative symptoms include, without limitation, lack of emotion, pleasure, motivation and desire to form relationships.

In some embodiments, the parenterally administering step is conducted subcutaneously or intramuscularly. These administration modes allow for long acting release and effectiveness. Dextromethorphan or similar pharmacological agent may be administered as a long acting injectable (as done with Haldol decanoate, Prolixin decanoate, Abilify Maintenna, and the like) in, for example, an oil-based vehicle, such as sesame seed oil, or with carboxymethyl cellulose and/or mannitol or micro-encapsulated in 75/25 polylactide-co-glycolide, or palmitate. Any subcutaneous or intramuscular carrier vehicle for DM may be used. In particular, extended or slow-release vehicles may be provided. Such vehicles may allow for less frequent injections which can assist in patient compliance. For resin based carriers, DM or similar pharmacological agent may be optionally conjugated and subsequently hydrolyzed to release DM or similar pharmacological agent at a controlled rate. The foregoing vehicles may allow for DM or similar pharmacological agent release over several weeks from a single injection. In other embodiments, DM or similar pharmacological agent may be administered as a bolus injection in the muscle or fat/subcutaneous tissue. DM or similar pharmacological agents may be slowly absorbed by the body from the depot injection. DM or similar pharmacological agents may have a continuous release over weeks and the serum level of dextromethorphan may be maintained for extended periods of time without another dosage administration. Continuous release formulations are particularly beneficial because compliance is an ongoing problem with treating schizophrenia patients. Moreover, continuous release methods may be useful to inhibit progression of the illness, since high glutamate levels cause death of central nervous system tissue. This is the most likely cause of schizophrenics having atrophy/loss of tissue in their brains.

In accordance with embodiments herein, quinidine is not administered to the subject when DM is administered via intramuscular or subcutaneous injection. This contrasts with oral administration, where quinidine is generally used. In some embodiments, the methods disclosed herein can further comprise co-administration (either simultaneous or sequentially in any order) of other active antipsychotic agents. In some embodiments, the methods further comprise administering haloperidol. In some embodiments, the methods further comprise administering fluphenazine. In some embodiments, the methods further comprise administering aripiprazole. For example, DM may be administered along with Haldol, Prolixin, Abilify, and the like as part of the same injection or as a secondary injection.

In some embodiments, a dosage of dextromethorphan is in a range from about 20 mg/kg/day to about 100 mg/kg/day. Those skilled in the art will recognize that an optimal dose needed in schizophrenia will depend on a number of factors and the values may be less than 20 mg/kg/day or greater than 100 mg/kg/day of dextromethorphan depending on patient characteristics. This range is merely what may be generally beneficial regardless of other patient attributes. Doses can be divided into multiple administrations, for example, twice daily or just single dose. Ideally, in injectable bolus form, these dosages refer to the release rate per day and the actual bolus dosage may be much higher and provide for extended release over days, weeks or even months. For example, injection range will depend on the vehicle, but may vary from about 100 mg to about 600 mg per month.

In some embodiments, there are provided methods of treating schizophrenia comprising intramuscularly or subcutaneously administering dextromethorphan to a subject in an amount from about 20 mg/kg/day to about 100 mg/kg/day, wherein the method avoids administering quinidine to the subject.

In some embodiments, there are provided methods of reducing symptoms associated with schizophrenia comprising intramuscularly or subcutaneously administering dextromethorphan to a subject in an amount from about 20 mg/kg/day to about 100 mg/kg/day, wherein the method avoids administering quinidine to the subject.

In some embodiments, there are provided methods of treating early onset schizophrenia comprising intramuscularly or subcutaneously administering dextromethorphan to a subject in an amount from about 20 mg/kg/day to about 100 mg/kg/day, wherein the method avoids administering quinidine to the subject.

In some embodiments, there are provided methods of slowing progression of schizophrenia comprising intramuscularly or subcutaneously administering dextromethorphan to a subject in an amount from about 20 mg/kg/day to about 100 mg/kg/day, wherein the method avoids administering quinidine to the subject.

In some embodiments, oral administration with DM-quinidine may be combined with injectable DM. In such embodiments, DM-quinidine may be administered first as a quick release agent, followed by injectable DM for maintenance.

In some embodiments, oral dextromethorphan may be administered in an amount from about 5 mg/day to about 100 mg/day. In some embodiments, the quinidine is administered in an amount from about 20 mg/day to about 60 mg/day. The range may be anywhere from 20 to 60 mg/day in equally divided doses to achieve optimal results in inhibiting the metabolism of dextromethorphan by the liver. In some embodiments, the dextromethorphan and quinidine are administered in amounts wherein the weight to weight ratio of dextromethorphan to quinidine is not greater than about 1:0.75. In some embodiments, the dextromethorphan and quinidine is administered as one combined dose per day. In some embodiments, the dextromethorphan and quinidine is administered as at least two combined doses per day.

In some embodiments, at least one of the dextromethorphan and quinidine is in a form of a salt selected from the group consisting of a salt of free acid, an inorganic salt, a salt of sulfate, a salt of hydrochloride, or a salt of hydrobromide. In some embodiments, the dextromethorphan is a decanoate salt.

In some embodiments, the symptoms of schizophrenia are caused by increased glutamate in the central nervous system. There are different types of causes based on a few factors. First, recent genetic studies have indicated that there are six different types of schizophrenia. Second, if it were a single illness, there should be some level of response in all patients. However, only one third of patients treated respond exceptionally, one third good, and one third not at all. In some embodiments, treating symptoms of schizophrenia comprises reducing negative symptoms.

Schizophrenia is not generally recognized to be occurring until after substantially altered behaviors in what is called a “psychotic break”, or “first break”. In some embodiments, the pharmacological agent is administered within 48 hours following said subject's first break. In some embodiments, the pharmacological agent is administered within 24 hours following said subject's first break. Schizophrenia first break is the first time symptoms worsen enough to necessitate hospitalization or require care by a psychiatrist. The earlier treatment is provided, the more likely the illness can be stopped from progressing and potentially the illness can even be reversed because there will be less atrophy/reduction of tissue in the brain. The full onset of schizophrenia is typically preceded by a gradual precursor period characterized by odd behavior and experiences, such as anxiety, restlessness and hallucinations. There may be a gradual loss of reality.

In some embodiments, methods herein may further comprise administering a neuroleptic. Exemplary neuroleptic agents include, without limitation, benperidol, bromperidol, droperidol, haloperidol, timiperone, fluspirilene, penfluridol, pimozide, acepromazine, chlorpromazine, cyamemazine, dixyazine, fluphenazine, levomeppromazine, perazine, pericyazine, perphenazine, pipotiazine, procholrperazine, promethazine, prothipendyl, thioproperazine, trifluoperazine, chlorprothixene, clopenthixol, flupentixol, thiothixene, zuclopenthixol, clotiapine, loxapine, prothipendyl, carpipramine, clocapramine, molindone, mosapramine, sulpride, sultopride, veralipride, amisuloride, amoxapine, aripiprazole, asenapine, cariprazine, bionanserin, iloperidone, lurasidone, melperone, nemonapride, olanzapine, paliperidone, perospirone, quetiapine, risperidone, sertindole, sultopride, trimipramine, ziprasidone, and zotapine.

In some embodiments, methods herein may further comprise administering a metabolic agent. In some embodiments, the metabolic agent may inhibit the metabolic rate of the pharmacological agent. The metabolic agent can be selected with reference to the pharmacological agent. For example, and without limitation, the pharmacological agent can comprise dextromethorphan and the metabolic agent can comprise quinidine. Metabolic agents that can reduce DM metabolism upon co-administration through blockade of the 2D6 system include, without limitation, amiodarone, celecoxib, chloroquine, chlorpromazine, cimetidine, citalopram, clomipramine, codeine, delavirdine, desipramine, destropropxyphene, diltiazem, doxorubicin, entaacapone, fluoxetine, fluphenazine, fluvoxamine, haloperidol, labetalol, lobeline, methadone, mibefradil, moclobomide, norfluoxetine, paroxetine, perphenazine, propafenone, quinacrine, quinidine, ranitidine, risperidone, ritonavir, sertindole, sertraline, thioridazine, valproic acid, venlafaxine, vinblastine, vincristine, vinorelbine and yohimbine.

When oral administration of DM/Q is combined with parenteral administration of DM, quinidine metabolism can be reduced by inhibitors of the CYP 3A4 system which include, without limitation, amiodarone, anastrozole, azithromycin, cannabinoids, cimetidine, clarithromycin, chlotrimazole, cyclosporine, danazol, delavirdine, dexamethasone, diethyldithiocarbamate, diltiazem, dirithvromycin, disulfiram, entacapone, erythromycin, fluconazole, fluoxetine, fluvoxamine, gestodene, grapefruit juice, indinavir, isoniazid, ketoconazole, metronidazole, mebenfradil, miconazole, nefazodone, nelfinavir, nevirapine, norfloxacin, omeprazole, paroxetine, propoxyphene, quinidine, quinine, quinupristine, ranitidine, ritonavir, saquinavir, sertindole, sertraline, trogiitazone, troleandomycin, and valproic acid.

In some embodiments, the pharmacological agent and/or metabolic agent can be administered orally. However it is to be appreciated that in accordance with some embodiments the pharmacological agent and/or metabolic agent can be administered subcutaneously, intravenously, intramuscularly, or by other means of administration. It is to be appreciated that when the metabolic agent is administered non-orally, the pharmacological agent can be administered without administration of a metabolic agent. In some embodiments, and in particular in some embodiment where the pharmacological agent is administered non-orally, the method can additionally include administering a fatty acid. For example, and without limitation, the pharmacological agent can comprise a decanoate salt or ester of dextromethorphan. It is to be appreciated that administration of the pharmacological agent may be via a “depot injection” such that an effective amount of dextromethorphan is continuously bioavailable to inhibit glutamate production.

In some embodiments, the metabolic agent can comprise quinidine. In some embodiments the quinidine can be administered in an amount from about 3 mg/day to about 75 mg/day. The quinidine may be in a form of a salt of free acid, an inorganic salt, a salt of sulfate, a salt of hydrochloride, or a salt of hydrobromide.

In some embodiments, the pharmacological agent may comprise dextromethorphan and the metabolic agent may comprise quinidine, where the effective amount of dextromethorphan can be from about 5 mg/day to about 100 mg/day, and the amount of quinidine can be from about 3 mg/day to about 75 mg/day. In some embodiments the weight-to-weight ratio of dextromethorphan to quinidine can be no greater than 1:0.75.

It is to be appreciated that a formulation of DM/Q has been approved for treatment of PBA and generalized to any neurological disorder where PBA is present. Specifically, the formulation of DM/Q available under the trademark NUEDEXTA® is an approved formulation of DM/Q. Thus, in some embodiments a method for treating symptoms of schizophrenia comprises administering the current formulation of DM/Q as NUEDEXTA®. This formulation comprises 20 mg dextromethorphan hydrobromide (morphinan, 3-methoxy-17-methyl-, (9a, 13a, 14a)- hydrobromide monohydrate) and 10 mg quinidine sulfate (cinchonan-9-ol, 6′-methoxy-(9S) sulfate (2:1), (salt), dihydrate). In some embodiments, this formulation may be administered between 1 and 5 times per day. Such oral administration may provide a period of treatment prior to parenteral administration, the latter providing longer term maintenance.

The following Examples show the immediate benefits of oral DM/Q in the management of schizophrenia.

### Example 1: Treatment of Subject suffering a Stroke

A subject with schizophrenia who was also known to have had a stroke was admitted to the hospital for worsening psychosis, aggression, paranoia, and unpredictable behaviors. The subject's negative symptoms of schizophrenia predated his pseudobulbar affect symptoms. Conventional theory that increasing glutamate can attenuate symptoms of schizophrenia, as in the case of a stroke where glutamate levels typically increase, could not explain the worsening schizophrenia symptoms (conventional theory would suggest that the subject's symptoms would have not worsened, but improved or stayed the same by increasing glutamate levels). The subject was orally administered DM/Q which not only resolved the subject's anger and impulsive behaviors, but also improved the subject's negative symptoms of schizophrenia. The subject began to laugh appropriately, pick up on social cues, joke appropriately, groom, and have a marked reduction in hostility. There were no side effects from the DM/Q. Administration to this subject of 20 mg dextromethorphan and 10 mg quinidine given orally twice daily was found to markedly improve negative symptoms of schizophrenia within 24 to 48 hours. Improvement in the subject's psuedobulbar affect and negative symptoms continued as long as the subject was administered DM/Q.

### Example 2: Treatment following First Break

Three subjects were presented to mental health following the initial onset of schizophrenic symptoms (“first break schizophrenia”). Each subject was administered DM/Q and a neuroleptic. Two subjects received aripiprazole as a neuroleptic and one received risperidone. Two of the subjects had a resolution of their illness to a point where they more closely approximated the behaviors seen in non-afflicted individuals (one on aripiprazole and one on risperidone). The illness recurred in both individuals when they stopped the DMQ on their own. This happened in one patient after 3 months and another after 10 months. Benefit was restored when the medication was reintroduced. This Example supports use of DM/Q to prevent the progression of schizophrenia in early onset, and also indicates that early use of DM/Q may mitigate the progression of schizophrenia along the entire course of the illness. Since glutamate is known to have neurotoxicity if levels are too high, the reduction in negative symptoms of schizophrenia arising from administration of DM/Q appears to reduce glutamate levels which increase following first break schizophrenia, thus interfering with the mechanism through which schizophrenia advances.

### Example 3: Additional Treatments

Oral administration of DM/Q in over 60 other schizophrenic subjects with treatment refractory illness led to a statistically large improvement in negative symptoms for the majority of them. The vast majority showed a significant improvement in negative symptoms, as well as a reduction in hostility (usually viewed as a positive symptom of schizophrenia). Not a single subject worsened on the medication, and careful scrutiny of the nursing and physician notes indicated no side effects of note in the schizophrenic population under investigation. Analysis of the data showed about 40% of the group improving markedly, 30% well enough to justify continuing the treatment protocol, and 20% showing little improvement.

Administration to acute and chronic schizophrenic subjects of a pharmacological agent that inhibits presynaptic glutamate release and/or modulates postsynaptic glutamate response, and in particular, dextromethorphan, can be used to treat symptoms of schizophrenia, and in particular, negative symptoms. Oral administration of 20 mg dextromethorphan and 10 mg quinidine per day can reduce negative symptoms of schizophrenia.

It is to be understood that variations and/or modifications of the present embodiments may be made without departing from the scope thereof. It is also to be understood that the present embodiments are not to be limited by the specific descriptions, or illustrations or combinations of either components or steps disclosed herein. Rather it is to be appreciated that these embodiments, descriptions, and illustrations are exemplary and are not meant to be limiting in scope.

What is claimed is:

1. A method of treating schizophrenia comprising parenterally administering to a subject a pharmacological agent that inhibits presynaptic glutamate release, modulates postsynaptic glutamate response, or both.

2. The method of claim 1, wherein treating schizophrenia comprises modulating positive symptoms.

3. The method of claim 1, wherein treating of schizophrenia comprises modulating negative symptoms.

4. The method of claim 1, wherein the parenterally administering step is conducted subcutaneously or intramuscularly.

5. The method of claim 1, wherein quinidine is not administered to the subject.

6. The method of claim 1, wherein the pharmacological agent comprises dextromethorphan.

7. The method of claim 1, further comprising administering haloperidol, fluphenazine, or aripiprazole.

8. The method of claim 6, wherein a dosage of dextromethorphan is in a range from about 20 mg/kg/day to about 100 mg/kg/day.

9. The method of claim 1, wherein the pharmacological agent is administered within 48 hours following the subject's first break.

10. The method of claim 1, further comprising administering a metabolic agent.

11. The method of claim 1, further comprising administering a neuroleptic.

12. The method of claim 1, further comprising orally administering dextromethorphan and quinidine.

13. The method of claim 12, wherein the orally administering step precedes parenteral administration.
